(19) United States
(12) Patent Application Publication
(10) Pub. No.: US 2024/0254384 A1
(43) Pub. Date: Aug. 1, 2024

(54) METHOD FOR OBTAINING BIO-SOURCED (METH)ALLYLSULFONATE ALKALI SALT

(71) Applicant: SNF Group, ANDREZIEUX BOUTHEON (FR)

(72) Inventors: Cédrick FAVERO, ANDREZIEUX BOUTHEON (FR); Johann KIEFFER, ANDREZIEUX BOUTHEON (FR)

(21) Appl. No.: 18/562,598

(22) PCT Filed: Jul. 8, 2022
(86) PCT No.: PCT/EP2022/069150

§ 371 Date: Nov. 20, 2023

(30) Foreign Application Priority Data
Jul. 9, 2021 (FR) 2107505
Jul. 9, 2021 (FR) 2107506

(51) Int. Cl. C09K 8/88; C09K 8/42

(52) U.S. Cl. CPC C09K 8/882; C09K 8/424

(57) ABSTRACT

A method is for obtaining a bio-sourced (meth)allylsulfonate alkali salt obtained from a (meth)allyl halide. The (meth)allyl halide is at least partially renewable and non-fossil. A (co)polymer can be obtained from the bio-sourced (meth)allylsulfonate alkali salt. The (co)polymer can be used in a method for enhanced oil and/or gas recovery by sweeping a subterranean formation.

### FIELD OF THE INVENTION

The present invention relates to a method for obtaining a bio-sourced (meth)allylsulfonate alkali salt (also called bio-sourced-(meth)allylsulfonate alkali salt) obtained from a (meth)allyl halide, said (meth)allyl halide being at least partially renewable and non-fossil. The invention also relates to a bio-sourced polymer obtained from at least one of bio-sourced (meth)allylsulfonate alkali salt according to the invention. Lastly, the invention relates to the use of the invention's bio-sourced polymers in various technical fields.

### PRIOR ART

Sodium (meth)allylsulfonate is a monomer widely used in manufacturing water-soluble or cross-linked polymers, and it is important to have a high quality sodium (meth)allylsulfonate with the lowest possible level of impurities.

The sodium allylsulfonate monomer can be obtained from a (meth)allyl halide on which sodium sulfite is reacted. As an example, we will cite document U.S. Pat. No. 4,171,324 which describes this reaction:

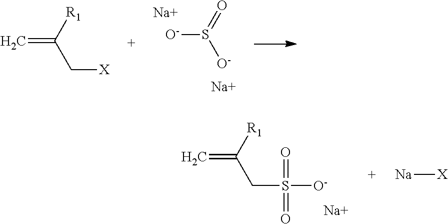

Where R₁═H or CH₃, preferentially H, and X═Cl, Br, I, preferentially Cl.

Document CN 109232329 also describes the synthesis of sodium allyl sulphonate (SAS) from sodium metabisulphite, sodium hydroxide and allyl chloride. However, it does not specify that the reagents should be of bio-sourced origin.

Document WO 2018/059745 describes bio-sourced allylic alcohol. It does not relate to the synthesis of sodium allyl sulphonate.

Document WO 2016/202894 describes a process for preparing an alcohol halide by reacting an alcohol and an aromatic carboxylic acid halide, in the presence of an N-substituted formamide. It does not specify that the reagents should be of bio-sourced origin.

Werpy et al. (“Top Value Added Chemicals From Biomass Volume I—Results of screening for potential candidates from sugars and synthesis gas” produced by the Staff at PNLL, NREL and EERE for the Office of the biomass August 2004) have identified products that can originate from biomass. The authors do not describe the synthesis of SAS.

(Meth)allyl chloride is produced by chlorination of propylene (CH₂═CH—CH₃) as described in document EP 0 455 644. Propylene is a fossil-based olefin, and is currently produced by steam cracking of naphtha, itself derived from crude oil refining. More recently, and with the advent of shale gas production, various propane dehydrogenation methods to produce propylene have been described.

Fossil-based propylene contains various impurities, which remain or are transformed in the method for producing (meth)allyl chloride and thus sodium (meth)allylsulfonate.

The impurities present in fossil-based sodium (meth)allyl are present in the sodium (meth)allylsulfonate monomer. They may even react during the manufacturing method of this monomer and thus generate new impurities, particularly where the (meth)allyl alkali salt contains saturated alkyl alkali salts, alkylene alkali salts containing at least two unsaturations and the hydrolysis products of alkyl and alkylene alkali salts. These impurities are known to limit the molecular weight of polymers incorporating sodium (meth)allylsulfonate or to induce unwanted cross-links.

Consequently, impurities present in (meth)allyl halide will induce an impaired performance of water-soluble polymers incorporating sodium (meth)allylsulfonate, either through molecular weight limiting effects, or through the presence of branched or cross-linked polymers.

The problem the invention proposes to resolve is to provide a new and improved method for producing (meth)allylsulfonate alkali salt.

### SUMMARY OF THE INVENTION

Quite surprisingly, the Applicant has observed that the use of a compound that is least partially renewable and non-fossil in a method for obtaining (meth)allylsulfonate alkali salt, allows to significantly improve the quality of the monomer obtained, and thus to improve the polymerization and the application performance of the polymers.

Without seeking to be bound by any particular theory, the Applicant raises the possibility that the different nature of the impurities between fossil-based compound and a renewable and non-fossil-based compound is the cause of these unexpected technical effects.

The very first object of the invention is a method for obtaining bio-sourced (meth)allylsulfonate alkali salt from (meth)allyl halide, said (meth)allyl alkali salt being at least partially renewable and non-fossil.

Another object of the invention is a bio-sourced (meth)allylsulfonate alkali salt obtained according to the method according to the invention, and a bio-sourced (meth)allylsulfonate alkali salt that is at least partially renewable and non-fossil.

Another object of the invention is a polymer obtained by polymerization of at least one bio-sourced (meth)allylsulfonate alkali salt obtained by the method according to the invention, or obtained by polymerization of at least one bio-sourced (meth)allylsulfonate alkali salt according to the invention. A further object of the invention is using the polymer according to the invention in various technical fields.

With the present invention, it is possible to achieve environmental objectives inherent in new technical innovations. It also allows to obtain polymerizable bio-sourced-monomers which deliver unexpectedly improved performances.

The renewable and non-fossil origin of allyl halide in a method for obtaining (meth)allylsulfonate alkali salt (synthesis involving a (meth)allyl halide and a sulfite ion precursor SO₃²⁻) allows 1) reducing the amount of SO₃²⁻ residues and 2) limiting the presence/formation of (meth)allyl alcohol.

Additionally, polymers and copolymers of bio-sourced-(meth)allylsulfonate alkali salt are more effective as agents for inhibiting scales (dispersant properties) than polymers of non-bio-sourced (meth)allylsulfonate alkali salt. Without wishing to be bound by any theory, the Applicant puts forward the possibility that the different nature of the impurities between a compound of fossil origin and a compound of renewable and non-fossil origin is the cause of these unexpected technical effects.

### DETAILED DESCRIPTION OF THE INVENTION

In the context of the invention, the terms “renewable and non-fossil” are used to designate the origin of a chemical compound derived from biomass or from synthesis gas (syngas), i.e. resulting from one or more chemical transformations carried out on one or more natural and non-fossil raw materials. The terms “bio-sourced” or “bio-resourced” can also be used to characterize the renewable and non-fossil origin of a chemical compound. The renewable and non-fossil origin of a compound includes renewable and non-fossil raw materials stemming from the circular economy, which have been previously recycled, once or several times, in a biomass material recycling process, such as materials from polymer depolymerization or pyrolysis oil processing.

According to the invention, the “at least partially renewable and non-fossil” quality of a compound means a bio-sourced carbon content preferably between 5 wt % and 100 wt % relative to the total carbon weight of said compound.

In the context of the invention, the ASTM D6866-21 standard Method B is used to characterize the bio-sourced nature of a chemical compound and to determine the bio-sourced carbon content of said compound. The value is expressed as a weight percentage (wt %) of bio-sourced carbon relative to the total carbon weight in said compound.

The ASTM D6866-21 standard is a test method that teaches how to experimentally measure the bio-sourced carbon content of solids, liquids and gaseous samples by radiocarbon analysis.

This standard primarily uses Accelerator Mass Spectrometry (AMS) technology. This technique is used to naturally measure the radionuclides present in a sample, wherein the atoms are ionized, then accelerated to high energies, then separated, and individually counted in Faraday cups. This high-energy separation is extremely effective at filtering out isobaric interference, so that AMS is able to accurately measure abundances of carbon-14 relative to carbon-12 (14C/12C) to an accuracy of 1·10⁻¹⁵.

The ASTM D6866-21 standard Method B uses AMS and IRMS (Isotope Ratio Mass Spectroscopy). The test method allows to directly differentiate contemporary carbon-based carbon atoms from fossil-based carbon atoms. A measure of the carbon-14 to carbon-12 or carbon-14 to carbon-13 content of a product is determined against a modern carbon-based reference material accepted by the radiocarbon dating community such as the NIST's Standard Reference Material (SRM) 4990C (oxalic acid).

The sample preparation method is described in the standard and does not require any special comment as it is a commonly used procedure.

Analysis, interpretation and reporting of results are described below. Isotope ratios of carbon-14 to carbon-12 content or carbon-14 to carbon-13 content are measured using AMS. Isotope ratios of carbon-14 to carbon-12 content or carbon-14 to carbon-13 content are determined relative to a standard traceable via the NIST SRM 4990C modern reference standard. The “fraction of modern” (fM) represents the amount of carbon-14 in the tested product relative to the modern standard. It is most often referred to as percent modern carbon (pMC), the percentage equivalent to fM (e.g. fM 1=100 pMC).

All pMC values obtained from radiocarbon analyses must be corrected for isotopic fractionation using a given stable isotope. The correction should be made using the carbon-14 to carbon-13 values determined directly using the AMS where possible. If this is not possible, the correction should be made using the delta 13C (δ13C) measured by IRMS, CRDS (Cavity Ring Down Spectroscopy) or any other equivalent technology that can provide accuracy to within plus or minus 0.3 per thousand.

“Zero pMC” represents the total absence of measurable 14C in a material above the background signals, thus indicating a fossil (e.g. petroleum-based) carbon source. A value of 100 pMC indicates a fully “modern” carbon source. A pMC value between 0 and 100 indicates a proportion of carbon derived from a fossil source relative to a “modern” source.

The pMC may be higher than 100% due to the persistent, but diminishing, effects of 14C injection into the atmosphere caused by atmospheric nuclear testing programmes. The pMC values need to be adjusted by an atmospheric correction factor (REF) to obtain the actual bio-sourced content of the sample.

The correction factor is based on the excess 14C activity in the atmosphere at the time of testing. A REF value of 102 pMC was determined for 2015 based on CO₂ measurements in the air in a rural area of the Netherlands (Lutjewad, Groningen). The first version of this standard (ASTM D6866-04) in 2004 had referenced a value of 107.5 pMC, while the later version ASTM D6866-10 (2010) had referenced a value of 105 pMC. These data points represent a drop of 0.5 pMC per year. Consequently, on 2 January of each year, the values in Table 1 below were used as REF value until 2019, reflecting the same decrease of 0.5 pMC per year. The REF values (pMC) for 2020 and 2021 have been determined to be 100.0 based on continuous measurements in the Netherlands (Lutjewad, Groningen) until 2019. References for reporting carbon isotope ratio data are provided below for 14C and 13C, respectively Roessler, N., Valenta, R. J., and van Cauter, S., “Time-resolved Liquid Scintillation Counting”, Liquid Scintillation Counting and Organic Scintillators, Ross, H., Noakes, J. E., and Spaulding, J. D., Eds., Lewis Publishers, Chelsea, M I, 1991, pp. 501-511. Allison, C. E., Francy, R. J., and Meijer, H. A. J., “Reference and Intercomparison Materials for Stable Isotopes of Light Elements”, International Atomic Energy Agency, Vienna, Austria, IAEATECHDOC-825, 1995.

The percentage of the bio-sourced carbon content is calculated by dividing pMC by REF and multiplying the result by 100. For example, [102 (pMC)/102 (REF)]×100=100% bio-sourced carbon. The results are indicated as a weight percentage (wt %) of bio-sourced carbon relative to the total carbon weight in said compound.

TABLE 1
Reference of percentage of modern carbon (pMC)

| REF year | pMC |
|---|---|
| 2015 | 102.0 |
| 2016 | 101.5 |
| 2017 | 101.0 |
| 2018 | 100.5 |
| 2019 | 100.0 |
| 2020 | 100.0 |
| 2021 | 100.0 |

In the context of the invention, the term “segregated” means a material stream that is distinctive and distinguishable from other material streams in a value chain (e.g. in a product manufacturing method), and thus considered to belong to a set of materials having an equivalent nature, such that the same origin of the material, or its manufacture according to the same standard or norm, can be tracked and guaranteed throughout this value chain.

For example, this may be the case of a chemist buying 100% bio-sourced (meth)allyl halide exclusively from a single supplier who guarantees the 100% bio-sourced origin of the (meth)allyl halide delivered, and the separate processing of other potential sources of (meth)allyl halide by said chemist converting this 100% bio-sourced (meth)allyl halide to produce a chemical compound. If the chemical compound produced is made solely from said 100% bio-sourced (meth)allyl halide, then the chemical compound is 100% bio-sourced.

In the context of the invention, the term “non-segregated”, in contrast to the term “segregated”, is understood to mean a material stream that cannot be differentiated from other material streams in a value chain.

In order to better understand this notion of segregation, it is useful to recall some basics about the circular economy and its practical application in methods, especially chemical transformation.

According to the French Environment and Energy Management Agency (ADEME), the circular economy can be defined as an economic system of trade and production which, at all stages of the life cycle of products (goods and services), seeks to increase efficiency in the use of resources and to reduce the environmental impact while developing the well-being of individuals. In other words, it is an economic system devoted to efficiency and sustainability that minimizes waste by optimizing value generated by resources. It relies heavily on a variety of conservation and recycling practices in order to break away from the current more linear “take-make-dispose” approach.

In the field of chemistry, which is the science of transforming one substance into another, this translates into reusing material that has already been used to make a product. Theoretically, all chemicals can be isolated and therefore recycled separately from other chemicals. The reality, particularly in industry, is more complex and means that even when isolated, the compound cannot often be differentiated from the same compound originating from another source, thus complicating the traceability of the recycled material.

For this reason, various traceability models have been developed taking into account this industrial reality, thereby allowing users in the chemical industry to manage their material streams with full knowledge of the facts, and allowing end customers to understand and know in a simple way the origin of the materials used to produce an object or a commodity.

These models have been developed to build transparency and trust throughout the value chain. Ultimately, this allows end-users or customers to choose a more sustainable solution without having the ability themselves to control every aspect of the method, by knowing the proportion of a desired component (e.g. of a bio-sourced nature) in an object or commodity.

One such model is “segregation”, which we have defined earlier. Some known examples where this model applies are glass and some metals where it is possible to track material streams separately.

However, chemicals are often used in complex combinations, and separate cycles are very often difficult to implement, especially due to prohibitive costs and highly complex stream management, such that the “segregation” model is not always applicable.

Consequently, when it is not possible to differentiate between material streams, other models are applied, which are grouped together under the term “non-segregated” and which entail, for example, taking into account the proportion of a specific stream relative to other streams, without physically separating the streams. One example is the Mass Balance Approach.

The Mass Balance Approach involves accurately tracking the proportion of a category (e.g. “recycled”) relative to a whole in a production system in order to guarantee, on the basis of an auditable account ledger, a proportionate and appropriate allocation of the content of that category in a finished product.

For example, this may be the case of a chemist buying 50% bio-sourced (meth)allyl halide from a supplier who guarantees, according to the mass or weight balance approach, that in the (meth)allyl halide delivered, 50% of the (meth)allyl halide has a bio-sourced origin, and de facto 50% is not of bio-sourced origin, and the use by said chemist of this 50% bio-sourced (meth)allyl halide with another stream of 0% bio-sourced (meth)allyl halide, the two streams not being identifiable at some point during the production process, due to mixing for example. If the chemical compound produced is made from 50% bio-sourced 50 wt % guaranteed (meth)allyl halide, and 0% bio-sourced 50 wt % (meth)allyl halide, the chemical compound is 25% bio-sourced.

In order to guarantee the stated “bio-sourced” figures, for example, and to encourage the use of recycled raw materials in producing new products, a set of globally shared and standardised rules (ISCC+, ISO 14020) has been developed to reliably manage material streams.

In the context of the invention, the term “recycled” is understood to mean the origin of a chemical compound derived from a method for recycling a material considered as waste, i.e. resulting from one or more transformations carried out using at least one recycling method on at least one material generally considered as waste.

The term “water-soluble polymer” is understood to mean a polymer which gives a clear aqueous solution when dissolved by stirring at 25° C. and with a concentration of 20 g·L⁻¹ in water.

### Method According to the Invention

The present invention relates to a method for obtaining a bio-sourced (meth)allylsulfonate alkali salt obtained from (meth)allyl halide and a sulfite ion precursor SO₃²⁻, said (meth)allyl halide being at least partially renewable and non-fossil.

In the whole invention, (meth)allyl halide is preferentially chosen from formula (1) compounds.

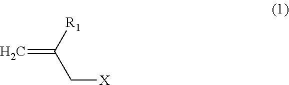

Where R₁═H or CH₃, preferentially H, and X═Cl, Br, or I, preferentially Cl.

The method according to the invention comprises a reaction between a (meth)allyl halide that is at least partially renewable and non-fossil with a sulfite ion precursor.

The (meth)allyl halide is preferentially a (meth)allyl chloride, but it can also be a (meth)allyl bromide or a (meth)allyl iodide.

The sulfite ion precursor is preferably sodium sulfite, but it can also be potassium sulfite, sodium bisulfite, potassium bisulfite, sodium metabisulfite, sodium dithionite, potassium metabisulfite, potassium dithionite, zinc dithionite, calcium dithionite, barium dithionite, magnesium dithionite.

When using sodium bisulfite or potassium bisulfite, the sulfite ion is generated in-situ or ex-situ by reaction with an alkaline base such as soda ash NaOH or potash KOH, for example. The sulfite ion precursor is preferentially sodium sulfite or sodium bisulfite, more preferentially sodium sulfite.

The monomer obtained according to the method is preferentially bio-sourced-sodium allylsulfonate, but it can also be bio-sourced sodium methallylsulfonate, bio-sourced-potassium allylsulfonate or bio-sourced potassium methallylsulfonate.

The bio-sourced (meth)allylsulfonate alkali salt is preferentially (meth)allylsulfonate sodium.

In the present description, the expressions “between X and Y” and “from X to Y” include the terminals X and Y.

In the whole invention, the bio-sourced carbon content of a compound for which it is specified that it is at least partially renewable and non-fossil, or for which the bio-sourced carbon content is specified, relative to the total carbon weight in said compound, ranges from 5 wt % to 100 wt %, and preferably from 10 wt % to 100 wt %, preferably from 15 wt % to 100 wt %, preferably from 20 wt % to 100 wt %, preferably from 25 wt % to 100 wt %, preferably from 30 wt % to 100 wt %, preferably from 35 wt % to 100 wt %, preferably from 40 wt % to 100 wt %, preferably from 45 wt % to 100 wt %, preferably from 50 wt % to 100 wt %, preferably from 55 wt % to 100 wt %, preferably from 60 wt % to 100 wt %, preferably from 65 wt % to 100 wt %, preferably from 70 wt % to 100 wt %, preferably from 75 wt % to 100 wt %, preferably from 80 wt % to 100 wt %, preferably from 85 wt % to 100 wt %, preferably from 90 wt % to 100 wt %, preferably from 95 wt % to 100 wt %, preferably from 97 wt % to 100 wt %, preferably from 99 wt % to 100 wt %, the bio-sourced carbon content being measured according to ASTM D6866-21 Method B.

In the invention and in the various embodiments described hereinafter, the bio-sourced (meth)allylsulfonate alkali salt preferentially has a bio-sourced carbon content ranging between 5 wt % and 100 wt % relative to the total carbon weight in said monomer, the bio-sourced carbon content being measured according to ASTM D6866-21 Method B.

In the invention and in the various embodiments described hereinafter, the (meth)allyl halide preferentially has a bio-sourced carbon content ranging between 5 wt % and 100 wt % relative to the total carbon weight in said (meth)allyl alkali salt, the bio-sourced carbon content being measured according to ASTM D6866-21 Method B.

In the invention and in the various embodiments described hereinafter, the (meth)allyl halide is preferentially totally renewable and non-fossil. The bio-sourced (meth)allylsulfonate alkali salt is preferentially totally renewable and non-fossil.

With respect to the reaction between the (meth)allyl halide and a sulfite ion precursor to form the (meth)allylsulfonate alkali salt, the person skilled in the art may refer to the already established knowledge.

The method is described hereinafter considering sodium sulfite and (meth)allyl chloride, but the method can be generalized with a sulfite ion precursor and a (meth)allyl halide.

In a first step, water and sodium sulfite, aqueous or anhydrous, is added into a reactor. In the case where solid sodium sulfite is used, the person skilled in the art will know how to adjust the amount of water to be added to have a sodium sulfite solution.

In a second step, (meth)allyl chloride is added, advantageously continuously, to the reactor for a time generally ranging between 1 minute and 24 hours, preferentially between 10 minutes and 12 hours, more preferentially between 30 minutes and 4 hours.

Simultaneously, an aqueous sodium hydroxide solution, preferentially at 50 wt % concentration, is added to the reactor, for a time preferentially ranging between 1 minute and 24 hours, more preferentially between 10 minutes and 12 hours., much more preferentially between 30 minutes and 4 hours. The amount of sodium hydroxide added is adjusted so as to maintain a pH of the reaction medium preferentially between 6 and 12, more preferentially between 8 and 10.

The molar ratio between the sodium sulfite and the (meth)allyl chloride is generally between 1:10 and 10:1, preferentially between 1:5 and 5:1, more preferentially between 1:2 and 1:1.5. The rate of addition of (meth)allyl chloride and/or sodium hydroxide can be constant, gradual or with any other profile.

The reaction medium's pH generally ranges between 6 and 12, preferentially between 8 and 10. The reaction can be carried out under atmospheric pressure or under pressure. In the latter case, the pressure is preferably between 1.1 bar absolute and 10 bar absolute. The reaction temperature is generally between 10 and 120° C., preferably between 50 and 90° C. The reaction can be conducted in batch, semi-batch or continuous mode.

At the end of the reaction, the sodium allylsulfonate is in aqueous solution in the presence of sodium chloride. The monomer product can be used as is, or undergo a separation step from the sodium chloride or a dehydration step. In this context, the person skilled in the art can refer to the already established knowledge.

In a non-limiting manner, and as an example, the sodium allylsulfonate solution can be distilled in order to evaporate the water. Sodium chloride then preferentially precipitates first, which can then be separated by filtration. If water evaporation is continued, the sodium allylsulfonate will then precipitate, and can be recovered in solid form after a filtration step.

In a non-limiting way, the filtration equipment can be a Nutsche filter, a filter press, a vertical or horizontal centrifuge, a rotary vacuum or pressure filter, or filtration can simply happen in the reactor if the latter is equipped at the drain with a grid with a suitable mesh to retain the sodium chloride and/or sodium allylsulfonate crystals.

Optionally, the aqueous solution of sodium allylsulfonate can be purified. In a non-limiting manner, the purification can be performed by distillation, evaporation on a falling film type equipment, a thin film evaporator or in a reboiler, by addition of steam, at atmospheric pressure or under vacuum.

The (meth)allyl halide may be non-segregated, partially segregated, or totally segregated.

Where the (meth)allyl halide is totally renewable and non-fossil, then it can be:
a) Either totally of recycled origin and
a)1) Or totally segregated;
a)2) Or partially segregated;
a)3) Or non-segregated;
b) Or partially of recycled origin and
b)1) Or totally segregated;
b)2) Or partially segregated;
b)3) Or non-segregated;
c) Or totally of non-recycled origin and
c)1) Or totally segregated;
c)2) Or partially segregated;
c)3) Or non segregated.

In these various embodiments, where the (meth)allyl halide is partially segregated, the weight ratio between the “segregated” part and the “non-segregated” part is preferably between 99:1 and 10:90, preferably between 99:1 and 30:70, or more preferably between 99:1 and 50:50.

Among these various embodiments, preference is given to the three a) embodiments, the three b) embodiments, and embodiment c)1). Among these embodiments, much greater preference is given to embodiments a)1), a)2), b)1), b)2) and c)1). The two most preferred embodiments are a)1) and b)1).

The industrial reality is such that it is not always possible to obtain industrial quantities of (meth)allyl halide that are bio-sourced, totally recycled and/or segregated or highly recycled and segregated. Hence, the above preferences may be more difficult to implement at the moment. From a practical standpoint, embodiments a)3), b)3), and c) are currently implemented more easily and on a larger scale. With techniques evolving quickly towards the circular economy, there is no doubt that the already applicable preferred modes will soon be applicable on a very large scale.

Where the (meth)allyl halide is/are partially renewable and non-fossil, a distinction is made between the renewable part (bio-sourced) and the non-bio-sourced part. Obviously, each of these parts can be according to the same embodiments a), b) and c) described hereinabove.

As concerns the bio-sourced part of the partially bio-sourced (meth)allyl halide, the same preferences apply as in the case where the compound is fully bio-sourced.

However, as concerns the non-bio-sourced part of the partially bio-sourced (meth)allyl halide, it is even more preferable to have as large a recycled component as possible for a circular economy approach. Hence, in this case, preference is given to embodiments a)1), a)2), b)1), b)2), particularly a)1) and b)1).

In a particular embodiment applicable to the various methods described in the invention, the (meth)allyl halide used in the method is partially or totally derived from a recycling process.

In this particular embodiment, the bio-sourced (meth)allyl halide is obtained using a recycling method, such as from polymer depolymerization or by manufacturing from pyrolysis oil, the latter resulting from high-temperature, anaerobic combustion of used plastic waste. Thus, materials considered as waste can be used as a source to produce recycled (meth)allyl halide, which in turn can be used as raw material to manufacture the invention's monomer. Since the monomer according to the invention is derived using a recycling method, the polymer according to the invention hereinafter described can cater to the virtuous circle of the circular economy.

In this particular embodiment, the method according to the invention comprises the following steps:
Recycling at least one material that is at least partially renewable and non-fossil to obtain (meth)allyl halide;
Converting said (meth)allyl halide into bio-sourced (meth)allylsulfonate alkali salt according to one of the previously described methods.

The recycling rate is the weight ratio of the recycled material to the total material.

In this particular embodiment, the part obtained from recycling is preferably totally “segregated”, i.e. is obtained from a separate pipeline and is treated in a separate manner. In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50.

### Monomer According to the Invention

The invention relates to a bio-sourced (meth)allylsulfonate alkali salt obtained according to one of the previously described methods. The invention also relates to a bio-sourced (meth)allylsulfonate alkali salt, being at least partially renewable and non-fossil. The same embodiments and preferences developed in the “methods” section apply to this section describing the monomer.

In the invention and in the various embodiments described hereinafter, the bio-sourced (meth)allylsulfonate alkali salt preferentially has a bio-sourced carbon content ranging between 5 wt % and 100 wt % relative to the total carbon weight in said monomer, the bio-sourced carbon content being measured according to ASTM D6866-21 Method B.

The bio-sourced (meth)allylsulfonate alkali salt is preferentially totally renewable and non-fossil.

The bio-sourced (meth)allylsulfonate alkali salt is preferentially obtained according to the various embodiments of the method previously described in the “method” section.

In the whole invention, bio-sourced (meth)allylsulfonate alkali salt, more generally bio-sourced monomer, is understood to mean a (meth)allylsulfonate alkali salt monomer or a monomer that is at least partially, preferentially totally, derived from biomass or synthesis gas (syngas), i.e. being the result of one or more chemical transformations carried out on one or more raw materials having a natural, as opposed to fossil, origin. The bio-sourced-(meth)allylsulfonate alkali salt can also be called bio-sourced or bio-resourced (meth)allylsulfonate alkali salt.

The (meth)allyl halide used to produce the bio-sourced (meth)allylsulfonate alkali salt may be non-segregated, partially segregated, or totally segregated. The same embodiments and preferences developed in the “methods” section apply to this section describing the monomer.

In a specific embodiment, the (meth)allyl halide used to produce the bio-sourced (meth)allylsulfonate alkali salt may be partially or totally recycled. The same embodiments and preferences developed in the “methods” section apply to this section describing the monomer.

### Polymer According to the Invention

The invention relates to a polymer obtained by polymerization of at least one bio-sourced (meth)allylsulfonate alkali salt obtained by the method according to the invention. It also relates to a polymer obtained by polymerization of at least one bio-sourced (meth)allylsulfonate alkali salt as previously described. The same embodiments and preferences developed in the “methods” section apply to this section describing the polymer.

The polymer according to the invention has the advantage of being partially or completely bio-sourced.

The polymer according to the invention is preferably water-soluble or water-swellable. The polymer may also be a superabsorbent.

The polymer according to the invention may be a homopolymer or a copolymer with at least one bio-sourced (meth)allylsulfonate alkali salt obtained by the method according to the invention, or as previously described, and with at least one different additional monomer, the latter advantageously being chosen from at least one nonionic monomer, and/or at least one anionic monomer, and/or at least one cationic monomer, and/or at least one zwitterionic monomer, and/or at least one monomer comprising a hydrophobic grouping.

Thus, the copolymer may comprise at least a second monomer different from the first monomer according to the invention, this second monomer being chosen from nonionic monomers, anionic monomers, cationic monomers, zwitterionic monomers, monomers comprising a hydrophobic grouping, and mixtures thereof.

The nonionic monomer is preferably selected from acrylamide, methacrylamide, N-isopropylacrylamide, N,N-dimethylacrylamide, N,N-diethylacrylamide, N-methylolacrylamide, N-vinylformamide (NVF), N-vinylacetamide, N-vinylpyridine and N-vinylpyrrolidone (NVP), N-vinyl imidazole, N-vinyl succinimide, acryloyl morpholine (ACMO), acryloyl chloride, glycidyl methacrylate, glyceryl methacrylate, and diacetone acrylamide.

The anionic monomer is preferably chosen from acrylic acid, methacrylic acid, itaconic acid, crotonic acid, maleic acid, fumaric acid, acrylamido undecanoic acid, 3-acrylamido 3-methylbutanoic acid, maleic anhydride, 2-acrylamido-2-methylpropane sulfonic acid (ATBS), vinylsulfonic acid, vinylphosphonic acid, allylsulfonic acid, methallylsulfonic acid, 2-sulfoethylmethacrylate, sulfopropylmethacrylate, sulfopropylacrylate, allylphosphonic acid, styrene sulfonic acid, 2-acrylamido-2-methylpropane disulfonic acid, and the water-soluble salts of these monomers, such as their alkali metal, alkaline earth metal or ammonium salts. It is preferably acrylic acid (and/or a salt thereof), and/or ATBS (and/or a salt thereof).

Preferably, the anionic monomer is maleic anhydride of its hydrated form, maleic acid.

Preferably, the maleic anhydride and/or its hydrated form, maleic acid, is at least partly of renewable and non-fossil origin.

The polymer according to the invention may be copolymer of 1) at least one bio-sourced-(meth)allylsulfonate alkali salt and 2) maleic anhydride and/or its hydrated form (maleic acid).

A process for obtaining maleic anhydride and/or its hydrated form, maleic acid, is either from 1) an alcohol, or from 2) a carboxylic acid or 3) from an alkene, said alcohol, carboxylic acid or alkene being preferably at least partly of renewable and non-fossil origin.

It is preferably a bio-sourced-maleic anhydride and/or its hydrated form thereof, maleic acid, which has a bio-sourced carbon content of between 5 wt % and 100 wt % relative to the total carbon weight in said compound, the bio-sourced carbon content being measured according to the standard ASTM D6866-21 Method B.

In the process for obtaining maleic anhydride and/or its hydrated form:
the alcohol has preferably a bio-sourced carbon content of between 5 wt % and 100 wt % relative to the total carbon weight in said alcohol, the bio-sourced carbon content being measured according to the standard ASTM D6866-21 Method B, and/or
the alcohol is preferably an alcohol comprising between 4 and 20 carbon atoms, preferentially the alcohol is N-butanol, 1,4-butanediol or tert-butanol, and/or
the carboxylic acid has preferably a bio-sourced carbon content of between 5 wt % and 100 wt % relative to the total carbon weight in said carboxylic acid, the bio-sourced carbon content being measured according to the standard ASTM D6866-21 Method B, and/or
the carboxylic acid is preferably succinic acid or a fatty acid preferentially comprising an aliphatic chain of 4 to 30 carbon atoms, and/or
the alkene has preferably a bio-sourced carbon content of between 5 wt % and 100 wt % relative to the total carbon weight in said alkene, the bio-sourced carbon content being measured according to the standard ASTM D6866-21 Method B, and/or
the alkene preferably comprises between 4 and 20 carbon atoms, preferentially the alkene is isobutylene, and/or
preferably, the carboxylic acid, alcohol or alkene is partially or totally segregated, and/or
preferably, the carboxylic acid, alcohol or alkene is partially or totally derived from a recycling process.

The cationic monomer is preferably chosen from quaternized dimethylaminoethyl acrylate (ADAME), quaternized dimethylaminoethyl methacrylate (MADAME), dimethyldiallylammonium chloride (DADMAC), acrylamido propyltrimethyl ammonium chloride (APTAC), and methacrylamido propyltrimethyl ammonium chloride (MAPTAC).

The zwitterionic monomer can be a derivative of a vinyl-type unit, particularly acrylamide, acrylic, allylic or maleic, this monomer having an amine or ammonium function (advantageously quaternary) and an acid function of the carboxylic (or carboxylate), sulfonic (or sulfonate) or phosphoric (or phosphate) type.

Monomers having a hydrophobic character can also be used in preparation of the polymer. Preferably, they are chosen from the group composed of esters of (meth)acrylic acid having an alkyl, arylalkyl, propoxylated, ethoxylated or ethoxylated and propoxylated chain; derivatives of (meth)acrylamide having an alkyl, arylalkyl, propoxylated, ethoxylated, ethoxylated and propoxylated chain, or dialkyl; alkyl aryl sulfonates, or of mono- or di-substituted amides of (meth)acrylamide having a propoxylated, ethoxylated, or ethoxylated and propoxylated alkyl, arylalkyl chain; derivatives of (meth)acrylamide having a propoxylated, ethoxylated, ethoxylated and propoxylated alkyl, arylalkyl, or dialkyl chain; alkyl aryl sulfonates

Each of these monomers may also be bio-sourced.

According to the invention, the polymer may have a linear, branched, star, comb, dendritic or block structure. These structures can be obtained by selecting the initiator, the transfer agent, the polymerization technique such as controlled radical polymerization referred to as RAFT (reversible addition-fragmentation chain transfer), NMP (Nitroxide Mediated Polymerization) or ATRP (Atom Transfer Radical Polymerization), incorporation of structural monomers, the concentration.

According to the invention, the polymer is advantageously linear and structured. A structured polymer refers to a non-linear polymer with side chains so as to obtain, when this polymer is dissolved in water, a pronounced state of entanglement leading to very substantial low gradient viscosities. The invention's polymer may also be cross-linked.

Additionally, the polymer according to the invention polymer may be structured:
By at least one structuring agent, which may be chosen from the group comprising polyethylenically unsaturated monomers (having at least two unsaturated functions), such as vinyl functions for example, particularly allyl, acrylic and epoxy functions, and one may mention, for example, methylene bis acrylamide (MBA), triallyamine, or tetraallylammonium chloride or 1,2 dihydroxyethylene bis-(N-acrylamide), and/or
By macroinitiators, such as polyperoxides, polyazoids and polytransfer agents, such as polymeric (co)polymers, and polyols, and/or
Functionalized polysaccharides.

The amount of branching/cross-linking agent in the monomer mixture is advantageously less than 4 wt % relative to the monomer content (weight), more advantageously less than 1%, and even more advantageously less than 0.5%. According to a particular embodiment, it may be at least equal to 0.00001 wt % relative to the monomer content.

In a particular embodiment, the polymer according to the invention may be a semi-synthetic and thus semi-natural polymer. In this embodiment, the polymer may be synthesized by copolymerization by total or partial grafting of at least one monomer according to the invention, and at least one natural compound, said natural compound being preferably chosen from starches and their derivatives, polysaccharides and their derivatives, fibers, vegetable gums, animal gums or algal gums, and modified versions thereof. For example, vegetable gums can include guar gum, gum arabic, locust bean gum, gum tragacanth, guanidinium gum, cyanine gum, tara gum, cassia gum, xanthan gum, ghatti gum, karaya gum, gellan gum, Cyamopsis tetragonoloba gum, soy gum, or beta-glucan or dammar. The natural compound can also be gelatin, casein, or chitosan. For example, algal gum can include sodium alginate or its acid, agar-agar, or carrageenan.

Polymerization is generally carried out, without this being limiting, by copolymerization or by grafting. The person skilled in the art will be able to refer to current general knowledge in the field of semi-natural polymers.

The invention also relates to a composition comprising at least one polymer according to the invention and at least one natural polymer, said natural polymer being preferably chosen from the previously described natural polymers. The weight ratio between the synthetic polymer and the natural polymer is generally between 90:10 and 10:90. The composition may be in liquid, inverse emulsion or powder form.

In general, the polymer does not require development of a particular polymerization method. Indeed, it can be obtained according to all the polymerization techniques well known to the person skilled in the art. In particular, it can be solution polymerization; gel polymerization; precipitation polymerization; emulsion polymerization (aqueous or inverse); suspension polymerization; reactive extrusion polymerization; water-in-water polymerization; or micellar polymerization.

Polymerization is generally free radical polymerization preferably by inverse emulsion polymerization or gel polymerization. Free radical polymerization includes free radical polymerization using UV, azo, redox or thermal initiators as well as controlled radical polymerization (CRP) techniques or matrix polymerization techniques.

The polymer according to the invention can be modified after it being obtained by polymerization. This is known as post-modification of the polymer. All known post-modifications can be applied to the polymer according to the invention, and the invention also relates to polymers obtained after said post-modifications. Among the possible post-modifications developed hereinafter, mention may be made of post-hydrolysis, post-modification by Mannich reaction, post-modification by Hoffman reaction and post-modification by glyoxalation reaction.

The polymer according to the invention can be obtained by performing a post-hydrolysis reaction on a polymer obtained by polymerization of at least one monomer obtained by the method according to the invention or at least one monomer as previously described in the “Monomer” section. Prior to post-hydrolysis, the polymer comprises acrylamide or methacrylamide monomer units, for example. The polymer may also further comprise monomeric units of N-Vinylformamide. More specifically, post-hydrolysis involves reaction of hydrolyzable functional groups of advantageously non-ionic monomeric units, more advantageously amide or ester functions, with a hydrolysis agent. This hydrolysis agent may be an enzyme, an ion exchange resin, an alkali metal, or a suitable acid compound. Preferably, the hydrolysis agent is a Brønsted base. Where the polymer comprises amide and/or ester monomer units, the post-hydrolysis reaction produces carboxylate groups. Where the polymer comprises vinylformamide monomer units, the post-hydrolysis reaction produces amine groups.

The polymer according to the invention can be obtained by performing a Mannich reaction on a polymer obtained by polymerization of at least one monomer obtained by the method according to the invention or at least one monomer as previously described in the “Monomer” section. More specifically, prior to the Mannich reaction, the polymer advantageously comprises acrylamide and/or methacrylamide monomer units. The Mannich reaction is performed in aqueous solution in the presence of a dialkyl amine and a formaldehyde precursor. More advantageously, the dialkyl amine is dimethylamine and the formaldehyde precursor is formaldehyde itself. After this reaction, the polymer contains tertiary amines.

The polymer according to the invention can be obtained by performing a Hoffman reaction on a polymer obtained by polymerization of at least one monomer obtained by the method according to the invention or at least one monomer as previously described in the “Monomer” section. Prior to the Hoffman reaction, the polymer advantageously comprises acrylamide and/or methacrylamide monomer units. The so-called Hofmann degradation reaction is carried out in aqueous solution in the presence of an alkaline earth and/or alkali hydroxide and an alkaline earth and/or alkali hypohalide.

Discovered by Hofmann at the end of the nineteenth century, this reaction is used to convert an amide function into a primary amine function with one carbon atom less. The detailed reaction mechanism is presented below.

In the presence of a Brønsted base (e.g., soda), a proton is extracted from the amide.

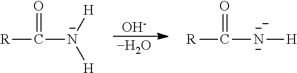

The amidate ion formed then reacts with the active chlorine (Cl₂) of the hypochlorite (e.g. NaClO which is in equilibrium: 2NaOH+Cl₂⇔NaClO+NaCl+H₂O) to produce an N-chloramide. The Brønsted base (e.g. NaOH) extracts a proton from the chloramide to form an anion. The anion loses a chloride ion to form a nitrene which undergoes isocyanate rearrangement.

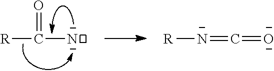

By reaction between the hydroxide ion and the isocyanate, a carbamate is formed.

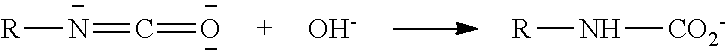

After decarboxylation (removal of CO₂) from the carbamate, a primary amine is obtained.

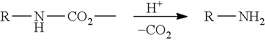

For the conversion of all or part of the amide functions of a (co)polymer comprising an amide group into an amine function, two main factors are involved (expressed in molar ratios). These are:
Alpha=(alkali and/or alkaline earth hypohalide/amide group) and
Beta=(alkali and/or alkaline earth hydroxide/alkali and/or alkaline earth hypohalide).

The polymer according to the invention can also be obtained by carrying out a glyoxalation reaction on a polymer obtained by polymerization of at least one monomer obtained by the method according to the invention or of at least one monomer as previously described in the “Monomer” section, said polymer comprising, with the glyoxalation reaction, at least one monomer unit advantageously of acrylamide or methacrylamide. More specifically, the glyoxalation reaction involves a reaction of at least one aldehyde on the polymer, thus allowing said polymer to be functionalized. Advantageously, the aldehyde may be chosen from the group comprising glyoxal, glutaraldehyde, furan dialdehyde, 2-hydroxyadipaldehyde, succinaldehyde, starch dialdehyde, 2.2 dimethoxyethanal, diepoxy compounds, and combinations thereof. Preferably, the aldehyde compound is glyoxal.

According to the invention, the polymer may be in liquid, gel or solid form when its preparation includes a drying step such as spray drying, drum drying, radiation drying, such as microwave drying, or fluid bed drying.

According to the invention, the water-soluble polymer preferably has a molecular weight between 1000 and 40 million g/mol. The polymer may be a dispersant (in particular anti-scaling agent), in which case its molecular weight is preferably between 1000 and 50.000 g/mol. The polymer may have a higher molecular weight, typically between 1 and 30 million g/mol. The molecular weight is understood as weight average molecular weight. The polymer according to the invention may also be a superabsorbent capable of absorbing from 10 to 500 times its weight in water.

The molecular weight is advantageously determined by the intrinsic viscosity of the (co)polymer. The intrinsic viscosity can be measured by methods known to the person skilled in the art and can be calculated from the reduced viscosity values for different (co)polymer concentrations by a graphical method entailing plotting the reduced viscosity values (y-axis) against the concentration (x-axis) and extrapolating the curve down to zero concentration. The intrinsic viscosity value is plotted on the y-axis or using the least squares method. The molecular weight can then be determined using the Mark-Houwink equation:

$$[ η ] = {KM}^{α}$$

[η] represents the intrinsic viscosity of the (co)polymer determined by the solution viscosity measurement method.
K represents an empirical constant.
M represents the molecular weight of the (co)polymer.
α represents the Mark-Houwink coefficient.
K and α depend on the specific (co)polymer-solvent system.

The co-monomers combined with the monomer according to the invention to obtain the polymer of the invention, are preferably at least partially, or more preferably totally renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention, and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention; and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil;
at least 1 mol %, preferably between 5 mol % and 90 mol %, more preferably between 10 mol % and 80 mol % of at least one third monomer comprising an ethylenic unsaturation, said third monomer being different from the first and the second monomers, and being at least partially renewable and non-fossil.

The polymer according to the invention may comprise four or more different monomers.

In a preferred embodiment, the second and the possible other monomers have a bio-sourced carbon content ranging between 5 wt % and 100 wt %, preferably 10 wt % and 100 wt %, relative to the total carbon weight in the related monomer, the bio-sourced carbon content being measured according to ASTM D6866-21 Method B.

In this preferred embodiment, the second and the possible other monomers are preferably chosen from acrylamide, acrylic acid and/or one of the salts thereof, an oligomer of acrylic acid, 2-acrylamido-2-methylpropane sulfonic acid (ATBS) and/or a salt thereof, N-vinylformamide (NVF), N-vinylpyrrolidone (NVP), dimethyldiallylammonium chloride (DADMAC) quaternized dimethylaminoethyl acrylate (ADAME), quaternized dimethylaminoethyl methacrylate (MADAME), a substituted acrylamide having the formula CH₂═CHCO—NR₁R₂, R₁ and R₂ being, independently of each other, a linear or branched carbon chain CₙH₂ₙ₊₁, wherein n is between 1 and 10.

In this preferred embodiment, alternatively, the second and the possible other monomers is maleic anhydride or its hydrated form, maleic acid.

In the whole invention, it will be understood that the molar percentage of the monomers (excluding any cross-linking agents) of the polymer is equal to 100%.

The (meth)allyl halide used to produce the bio-sourced (meth)allylsulfonate alkali salt may be non-segregated, partially segregated, or totally segregated. The same embodiments and preferences developed in the “methods” section apply to this section describing the polymer.

In a specific embodiment, the (meth)allyl halide used to produce the bio-sourced (meth)allylsulfonate alkali salt may be partially or totally recycled. The same embodiments and preferences developed in the “methods” section apply to this section describing the polymer.

In this particular embodiment, the invention relates to a polymer obtained according to a method comprising the following steps:
Recycling at least one material that is at least partially renewable and non-fossil to obtain (meth)allyl halide;
Converting said (meth)allyl halide into bio-sourced (meth)allylsulfonate alkali salt according to one of the previously described methods;
Polymerizing the bio-sourced (meth)allylsulfonate alkali salt, and/or optionally another unsaturated monomer.

The invention further relates to a polymer as previously described, comprising a bio-sourced carbon content of between 5 wt % and 100 wt % relative to the total carbon weight in said polymer, the bio-sourced carbon content being measured according to ASTM D6866-21 Method B.

The invention also relates to the use of at least one monomer obtained by the method according to the invention in order to synthesize a polymer.

### Using the Polymer According to the Invention

The invention also relates to the use of the polymer according to the invention in the recovery of hydrocarbons (oil and/or gas); in drilling and cementing of wells; in the stimulation of hydrocarbon wells (oil and/or gas), for example hydraulic fracturing, conformation, diversion; in the treatment of water in open, closed or semi-closed circuits; in the treatment of fermentation slurry, treatment of sludge; in paper manufacturing; in construction; in wood processing; in hydraulic composition processing (concrete, cement, mortar and aggregates); in the mining industry; in the formulation of cosmetic products; in the formulation of detergents; in textile manufacturing; in battery component manufacturing; in geothermal energy; in sanitary napkin manufacturing; or in agriculture.

The invention also relates to the use of the polymer according to the invention as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant (in particular anti-scaling agent).

### Method Using the Polymer According to the Invention

The present invention also relates to the various methods described hereinafter, wherein the polymers of the invention are used to improve application performance.

The invention also relates to a method for enhanced oil or gas recovery by sweeping a subterranean formation comprising the following steps:
a. Preparing an injection fluid from a polymer according to the invention with water or brine,
b. Injecting the injection fluid into a subterranean formation,
c. Sweeping the subterranean formation with the injected fluid,
d. Recovering an aqueous mixture of oil and/or gas.

The invention also relates to a method for hydraulic fracturing of subterranean oil and/or gas reservoirs comprising the following steps:
a. Preparing an injection fluid from a polymer according to the invention, with water or brine, and with at least one proppant,
b. Injecting said fluid into the subterranean reservoir and fracturing at least a portion thereof to recover oil and/or gas.

In the methods described hereinabove, the polymer is preferably a high molecular weight polymer (greater than 8 million daltons). It is preferably linear. It is preferably in the form of a powder, an inverse emulsion, a partially dehydrated inverse emulsion, or in the form of a “clear”, i.e. a dispersion of solid polymer particles in an aqueous or oily fluid. The powder form is preferably obtained by gel or spray drying of an inverse emulsion. It also involves a composition comprising an inverse emulsion of a polymer according to the invention and solid particles of a polymer according to the invention.

The invention also relates to a method of stimulation of a subterranean formation comprising the following steps:
a. Preparing an injection fluid from a polymer according to the invention with water or brine,
b. Injecting the injection fluid into a subterranean formation,
c. Partially or totally plugging the subterranean formation with the injected fluid, said plugging being temporary or permanent.

The invention also related a method of drilling and/or cementing a well in a subterranean formation comprising the following steps:
a. Preparing an injection fluid from a polymer according to the invention with water or brine,
b. Injecting said drilling and/or cementing fluid into the subterranean formation via the drill head in at least one step of drilling or cementing a well.

Drilling and cementing a well are two successive steps in creating a well in a subterranean formation. The first step is drilling with the drilling fluid, while the second step is cementing the well with the cementing fluid. The invention also relates to a method of injecting an intermediate fluid (“spacer fluid”) injected between the drilling fluid and the cementing fluid, said intermediate fluid comprising at least one polymer according to the invention. This intermediate fluid prevents contamination between the cementing fluid and the drilling fluid.

When drilling and cementing a well, the polymer according to the invention can be used as a fluid loss additive in well cement compositions in order to reduce fluid loss from the cement compositions to permeable formations or zones into or through which the cement compositions are pumped. In primary cementing, loss of fluid, i.e., water, to permeable formations or subterranean zones can lead to premature gelling of the cement composition, so that bridging the annular space between the permeable formation or zone and the drill string cemented therein prevents the cement composition from being placed along the entire length of the ring.

The invention also relates to a method of inerting clays in hydraulic compositions for construction purposes, said method comprising a step of adding to the hydraulic composition or one of its constituents at least one clay inerting agent, characterized in that the clay inerting agent is a polymer according to the invention.

Clays can absorb water and cause poor performance of building materials. When the polymer of the invention is used as a clay inhibitor, it allows in particular to avoid the clay swelling which may cause cracks thus weakening any building.

The hydraulic composition may be a concrete, cement, mortar or aggregate. The polymer is added to the hydraulic composition or to one of its constituents advantageously at a dosage of 2 to 200 ppm of inerting agent relative to the weight of aggregate.

In this method of inerting clays, clays include, but are not limited to, 2:1 swelling clays (such as smectite), or 1:1 swelling clays (such as kaolin) or 2:1:1 swelling clays (such as chlorite). The term “clay” generally refers to magnesium and/or aluminum silicate, including phyllo silicates with a lamellar structure. However, in the present invention, the term “clay” also includes clays having no such structure, such as amorphous clays.

The invention also relates to a method for manufacturing a sheet of paper, cardboard or the like, whereby, before a sheet is formed, a step is performed entailing adding to a suspension of fibers, at one or more injection points, at least one polymer according to the invention. The polymer may provide dry strength or retention properties or wet strength. It may also improve paper formation, drainage and dewatering capabilities.

The method can be used successfully to manufacture packaging papers and cardboards, coating papers, sanitary and household papers, any type of paper, cardboard or the like.

The post-modified polymers described in the “Polymers” section, in particular the post-modified polymers by Hoffman reaction or by glyoxalation reaction, are particularly advantageous in methods for manufacturing paper, cardboard or the like.

Retention properties are understood to mean the capability to retain the suspended materials of the paper pulp (fibers, fines, fillers (calcium carbonate, titanium oxide), . . . ) on the forming fabric, thus in the fibrous mat that will make up the final sheet. The mode of action of the retention agents is based on the flocculation of these suspended materials in water. Indeed, the flocs formed are more easily retained on the forming sheet.

The retention of fillers involves retaining specifically the fillers (small mineral species with little affinity with cellulose). Substantial improvement of retention of fillers leads to a clarification of white water by retaining the fillers in the sheet and by increasing its grammage. It also gives the possibility to replace part of the fibers (the most expensive species in the composition of paper, cardboard or similar) with fillers (lower costs) in order to reduce manufacturing costs.

As concerns dewatering (or drainage) properties, it is the capacity of the fibrous mat to evacuate or drain the maximum amount of water so that the sheet dries as quickly as possible, in particular during manufacturing of the sheet.

These two properties (retention and drainage) being intricately linked, one depending on the other, the issue is therefore to find the best compromise between retention and drainage. Generally, the person skilled in the art refers to a retention and drainage agent because these are the same types of products used to improve these two properties.

Fibrous suspension is understood to mean thick pulp or diluted pulp which are composed of water and cellulose fibers. The thick stock, with a dry matter concentration of more than 1% or even more than 3%, is located upstream of the fan pump. The thin stock, with a dry mass concentration of generally less than 1%, is located downstream of the fan pump.

The polymer can be added to the thick stock or to the thin stock. It can be added at the level of the fan pump or the headbox. Preferably, the polymer is added before the headbox.

In the method for making paper, cardboard or the like according to the invention, the polymer according to the invention may be used alone or in combination with a secondary retention agent.

Preferably, a secondary retention agent selected from organic polymers and/or inorganic microparticles is added to the fiber suspension.

This secondary retention agent added to the fibrous suspension is advantageously chosen from anionic polymers in the broad sense, which can therefore be (without being limiting) linear, branched, cross-linked, hydrophobic, associative and/or inorganic microparticles (such as bentonite, colloidal silica).

The invention also relates to a method for treating a suspension of solid particles in water resulting from mining or oil sands operations, comprising contacting said suspension with at least one polymer according to the invention. Such a method can be carried out in a thickener, which is a holding zone, generally in the form of a tube section of several meters in diameter with a conical bottom wherein the particles can settle. According to a specific embodiment, the aqueous suspension is transported by means of a pipe to a thickener, and the polymer is added to said pipe.

According to another embodiment, the polymer is added to a thickener that already contains the suspension to be treated. In a typical mineral processing operation, the suspensions are often concentrated in a thickener. This results in a higher density sludge that exits the bottom of the thickener, and an aqueous fluid released from the treated suspension (called liquor) that exits by overflow, from the top of the thickener. Generally, the addition of the polymer increases the concentration of the sludge and increases the clarity of the liquor.

According to another embodiment, the polymer is added to the particulate suspension during transport of said suspension to a deposition area. Preferably, the polymer is added in the pipe that conveys said suspension to a deposition zone. It is on this deposition area that the treated suspension is spread in preparation for dewatering and solidification. The deposition areas can be either open, such as an unconfined area of soil, or enclosed, such as a basin, cell.

An example of such treatments during transport of the suspension is spreading the suspension treated with the polymer according to the invention on the soil in preparation for dewatering and solidification and then spreading a second layer of treated suspension on top of the solidified first layer. Another example is the continuous spreading of the suspension treated with the polymer according to the invention in such a way that the treated suspension falls continuously on the suspension previously discharged in the deposition area, thus forming a mass of treated material from which water is extracted.

According to another embodiment, the water-soluble polymer is added to the suspension and a mechanical treatment is performed, such as centrifugation, pressing or filtration.

The water-soluble polymer can be added simultaneously in different stages of the suspension treatment, i.e., for example, in the pipe carrying the suspension to a thickener and in the sludge exiting the thickener which will be conveyed either to a deposition area or to a mechanical treatment device.

The invention also relates to a method for treating municipal or industrial water, comprising the introduction into said water to be treated of at least one polymer according to the invention.

Effective water treatment requires the removal of dissolved compounds, and dispersed and suspended solids from the water. Generally, this treatment is enhanced by chemicals such as coagulants and flocculants. These are usually added to the water stream ahead of the separation unit, such as flotation and sedimentation.

The polymers according to the invention can be advantageously used to coagulate or flocculate suspended particles in municipal or industrial wastewater. Generally, they are used in combination with inorganic coagulants such as alum.

They can also be used advantageously to treat the sludge produced from the treatment of this wastewater. Sewage sludge (be it urban or industrial) is the main waste produced by a treatment plant from liquid effluents. Generally, sludge treatment involves dewatering it. This dewatering can be performed by centrifugation, filter press, belt press, electro-dewatering, sludge drying reed beds, solar drying. It is used to decrease sludge water concentration.

In this municipal or industrial water treatment process, the polymer according to the invention is preferably linear or branched. It is preferably in the form of a powder, an inverse emulsion or a partially dehydrated inverse emulsion. The powder form is preferably obtained by gel or spray drying from an inverse emulsion.

The invention also relates to an additive for a cosmetic, dermatological or pharmaceutical composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to cosmetic, dermatological or pharmaceutical compositions comprising at least one polymer according to the invention.

In particular, reference may be made to application FR2979821 on behalf of L'OREAL for the manufacture of such compositions and description of the other ingredients of such compositions. The said compositions may be in the form of a milk, a lotion, a gel, a cream, a gel cream, a soap, a bubble bath, a balm, a shampoo or a conditioner. The use of said compositions for the cosmetic or dermatological treatment of keratinous materials, such as the skin, scalp, eyelashes, eyebrows, nails, hair and/or mucous membranes is also an integral part of the invention. Such use comprises application of the composition to the keratinous materials, possibly followed by rinsing with water.

The invention also relates to an additive for detergent composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to detergent compositions for household or industrial use comprising at least one polymer according to the invention. In particular, reference may be made to the applicant's application WO2016020622 for the manufacture of such compositions and description of the other ingredients of such compositions.

“Detergent compositions for household or industrial use” are understood to mean compositions for cleaning various surfaces, particularly textile fibers, hard surfaces of any kind such as dishes, floors, windows, wood, metal or composite surfaces. Such compositions include, for example, detergents for washing clothes manually or in a washing machine, products for cleaning dishes manually or for dishwashers, detergent products for washing house interiors such as kitchen elements, toilets, furnishings, floors, windows, and other cleaning products for universal use.

The polymer used as an additive, e.g., thickener, for a cosmetic, dermatological, pharmaceutical, or detergent composition is preferably cross-linked. It is preferably in the form of a powder, an inverse emulsion or a partially dehydrated inverse emulsion. The powder form is preferably obtained by spray drying from an inverse emulsion.

The invention equally relates to a thickener for pigment composition used in textile printing, said thickener comprising at least one polymer according to the invention. The invention also relates to a textile fiber sizing agent, said agent comprising at least one polymer according to the invention.

The invention also relates to a process for manufacturing superabsorbent from the monomer according to the invention, a superabsorbent obtained from at least one monomer according to the invention, said superabsorbent to be used for absorbing and retaining water in agricultural applications or for absorbing aqueous liquids in sanitary napkins. For example, the superabsorbent agent is a polymer according to the invention.

The invention also relates to a method for manufacturing sanitary napkins wherein a polymer according to the invention is used, for example as a superabsorbent agent.

The invention also relates to the use of the polymer according to the invention as a battery binder. The invention also relates to a battery binder composition comprising the polymer according to the invention, an electrode material and a solvent. The invention also relates to a method for manufacturing a battery comprising making a gel comprising at least one polymer according to the invention and filling same into said battery. Mention may be made of lithium ion batteries which are used in a variety of products, including medical devices, electric cars, aircraft and, most importantly, consumer products such as laptops, cell phones and cameras.

Generally, lithium ion batteries (LIBs) include an anode, a cathode, and an electrolyte material such as an organic solvent containing a lithium salt. More specifically, the anode and cathode (collectively, the “electrodes”) are formed by mixing an electrode active material (anode or cathode) with a binder and solvent to form a paste or sludge that is then applied and dried onto a current collector, such as aluminum or copper, to form a film on the current collector. The anode and cathode are then stacked and wound before being housed in a pressurized case containing an electrolyte material, all of which together form a lithium-ion battery.

In a lithium battery, the binder plays several important roles in both mechanical and electrochemical performance. Firstly, it helps disperse the other components in the solvent during the manufacturing process (some also act as a thickener), thus allowing for even distribution. Secondly, it holds the various components together, including the active components, any conductive additives, and the current collector, ensuring that all of these parts stay in contact. Through chemical or physical interactions, the binder connects these separate components, holding them together and ensuring the mechanical integrity of the electrode without a material impact on electronic or ionic conductivity. Thirdly, it often serves as an interface between the electrode and the electrolyte. In this role, it can protect the electrode from corrosion or the electrolyte from depletion while facilitating ion transfer across this interface.

Another important point is that the binders must have a certain degree of flexibility so that they do not crack or develop defects. Brittleness can create problems during manufacturing or assembly of the battery.

Given all the roles it plays in an electrode (and in the battery as a whole), choosing a binder is critical in ensuring good battery performance.

The invention also relates to a method for manufacturing sanitary napkins wherein a polymer according to the invention is used, for example as a superabsorbent agent.

As previously described, the circular economy is an economic system devoted to efficiency and sustainability that minimizes waste by optimizing value generated by resources. It relies heavily on a variety of conservation and recycling practices in order to break away from the current more linear “take-make-dispose” approach.

Therefore, with material recycling being a major and growing concern, recycling processes are developing rapidly and enabling the production of materials that can be used to produce new compounds or objects. Recycling materials does not depend on the origin of the material and as long as it can be recycled, it is considered as a technical progress. Although the origin of the material to be recycled may be renewable and non-fossil, it may also be fossil.

Specific objects are described hereinafter.

A first specific object relates to a method for obtaining (meth)allylsulfonate alkali salt obtained from (meth)allyl halide, said (meth)allyl halide being derived at least partially, preferentially totally from a recycling method of a renewable and non-fossil material, or from a fossil material.

The (meth)allyl halide is preferentially chosen from formula (1) compounds.

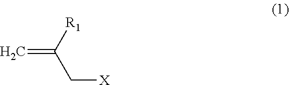

Where R₁═H or CH₃, preferentially H, and X═Cl, Br, or I, preferentially Cl.

The (meth)allyl halide is preferentially a (meth)allyl chloride, but it can also be a (meth)allyl bromide or a (meth)allyl iodide.

The sulfite ion precursor is preferably sodium sulfite, but it can also be potassium sulfite, sodium bisulfite, potassium bisulfite, sodium metabisulfite, sodium dithionite, potassium metabisulfite, potassium dithionite, zinc dithionite, calcium dithionite, barium dithionite, magnesium dithionite. When using sodium bisulfite or potassium bisulfite, the sulfite ion is generated in-situ or ex-situ by reaction with an alkaline base such as soda ash or potash.

The monomer obtained according to the method is preferentially sodium allylsulfonate, but it can also be sodium methallylsulfonate, potassium sodium allylsulfonate and potassium methallylsulfonate.

The (meth)allylsulfonate alkali salt is preferentially sodium (meth)allylsulfonate, more preferentially sodium allylsulfonate.

Preferentially, the (meth)allyl halide is totally “segregated”, i.e. from a separate pathway and treated separately. In an alternative embodiment, it is partially “segregated” and partially “non-segregated”. In this case, the weight ratio between the “segregated” part and the “non-segregated” part is preferentially between 99:1 and 25:75, preferably between 99:1 and 50:50. In an alternative embodiment, it is totally “non-segregated”.

A second specific object relates to a (meth)allylsulfonate alkali salt obtained from (meth)allyl alkali salt, said (meth)allyl alkali salt being derived at least partially, preferentially totally from a recycling method of a renewable and non-fossil material, or from a fossil material.

Another specific object relates to a polymer obtained by polymerization of at least one (meth)allylsulfonate alkali salt as just previously described.

A fourth specific object relates to the use of a polymer obtained by polymerization of at least one (meth)allylsulfonate alkali salt as just previously described, in the oil and/or gas recovery, in drilling and cementing of wells; in the stimulation of oil and/or gas wells (for example hydraulic fracturing, conformation, diversion), in the treatment of water in open, closed or semi-closed circuits, in the treatment of fermentation slurry, treatment of sludge, in paper manufacturing, in construction, in wood processing, in hydraulic composition processing (concrete, cement, mortar and aggregates), in the mining industry, in the formulation of cosmetic products, in the formulation of detergents, in textile manufacturing, in battery component manufacturing; in geothermal energy; or in agriculture.

A fifth specific object relates to the use of a polymer obtained by polymerization of at least one (meth)allylsulfonate alkali salt as just previously described as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

A sixth specific object relates to a polymer obtained according to a method comprising the following steps:
Recycling at least one material that is at least partially renewable and non-fossil to obtain (meth)allyl halide;
Converting said (meth)allyl halide into (meth)allylsulfonate alkali salt according to one of the previously described methods;
Polymerizing the (meth)allylsulfonate alkali salt, and optionally another unsaturated monomer.

Preferentially, the (meth)allylsulfonate alkali salt is totally “segregated”, i.e. from a separate pathway and treated separately.

In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50. In an alternative embodiment, it is totally “segregated”.

### EXAMPLES

The following examples relate to the synthesizing of bio-sourced sodium allyl sulfonate according to the invention (hereinafter abbreviated SAS), obtained from allyl chloride (hereinafter abbreviated ACL) and from a precursor of sulfite ions (hereinafter abbreviated SO₃²⁻). Said allyl chloride being at least partly of renewable and non-fossil origin. These examples also illustrate a copolymer obtained from these bio-sourced SAS, as well as its use as an antiscaling agent.

These examples best illustrate the advantages of the invention in a clear and non-limiting way.

### Description of the Method of Analysis in High Performance Liquid Chromatography.

The SAS purity is determined by high performance liquid chromatography under the following analysis conditions:

The purity of the SAS can be calculated by using external standards, and by measuring the areas of the different impurity peaks.

The quantification of allyl chloride and allyl alcohol is carried out via an external calibration of standards. Their retention time is calculated (Table 2).

TABLE 2

| Compound | Retention time (min) |
|---|---|
| Allyl chloride | 32.3 minutes |
| Allyl alcohol | 5.1 minutes |

### Description of the Sulfite Ion Analysis Method

The sulfite ions present in the SAS are measured by redox titration with an iodine reagent.

About 1 g of SAS solution is acidified with 33% concentrated hydrochloric acid to adjust the pH to between 6.5 and 7.5.

A Mettler T7 auto titrator, equipped with a pH and conductivity probe, is used for the measurement.

TABLE 3

| | Column filled with C₁₈ silica, | |
|---|---|---|
| | Zorbax Eclipse Plus C18 RRHT | |
| | type, with a diameter of 4.6 mm, | |
| | a length of 250 mm, and contains | |
| HPLC column characteristics | particles with a size of 10 μm | |
| Composition of | 80 wt % H₂O + | |
| the mobile phase: | 20 wt % methanol | |
| Injection volume: | 50 | μL |
| Column temperature: | 30° | C. |
| Injection flow rate: | 1 | mL/min |
| Detection wavelength: | 200 | nm |
| Analysis time: | 35 | minutes |

Experimental conditions for titration method:

TABLE 4

| Iodine I₂ concentration | 0.1N | |
|---|---|---|
| Maximum rate of | 0.5 | mL/min |
| iodine addition | | |
| Minimum rate of | 100 | L/min |
| iodine addition | | |
| Maximum Iodine Volume | 5 | mL |
| pH regulation | 7 +/− 2 | |
| pH regulation reagent | NaOH 1N | |
| Maximum rate of | 0.5 | mL/min |
| NaOH addition | | |
| Minimum rate of | 10 | μL/min |
| NaOH addition | | |
| Analysis time | ~5 | minutes |

The concentration of sulfite ions SO₃²⁻ is calculated according to the following formula:

$${[ sulfite ]}_{ppm} = \frac{[ I 2 ] ( mol . L ) * Veq ( L ) * Mw [ Na 2 SO 3 ] ( g . mol ) * 10 \hat{} 6}{[ sample ] ( g )}$$

### Example 1: Synthesis of Bio-Sourced Sodium Allyl Sulfonate

A test set is carried out according to the following protocol by adjusting the renewable and non-fossil origin of the allyl chloride and its percentage in ¹⁴C. (See Table 5)

The wt % of ¹⁴C is indicative of the nature of the carbon. The different levels of ¹⁴C are measured according to the ASTM D6866-21 standard, method B. This standard makes it possible to characterize the bio-sourced nature of a chemical compound by determining the bio-sourced carbon level of said compound. A “zero pMC” represents the total absence of measurable ¹⁴C in a material, thus indicating a fossil carbon source.

Allyl chloride of renewable and non-fossil origin comes from the treatment of residues from the paper pulp industry (“tall oil”), or agricultural waste, via bio-sourced-propylene. Allyl chloride can also come from the treatment of municipal waste, biomass, by fermentation or recycling of carbon dioxide or carbon monoxide. The fossil origin of allyl chloride comes from a fossil propylene

Three hundred eight g of demineralised water and 1362 g of a sodium bisulfite solution at 40% concentration by weight are added to a 2 500 mL autoclave equipped with a jacket, a stirrer and a condenser.

A first separating funnel is loaded with 391 g of sodium hydroxide concentrated at 50 wt %. A second separating funnel is loaded with 420 g of allyl chloride.

Nitrogen bubbling is carried out for 30 minutes to remove all traces of dissolved oxygen.

The autoclave is hermetically sealed. Sodium hydroxide and allyl chloride are added simultaneously to the reactor with pumps, over a period of 360 minutes. The reaction is exothermic and the temperature in the reactor is regulated at 50° C. The autogenous pressure is around 1.3 bars absolute.

After the addition of the reagents, the reaction medium is left under stirring for 2 hours in order to consume the residual sodium bisulfite. After 2 hours of reaction, the reactor is returned to atmospheric pressure.

In table 5, the applicant examines the synthesis of SAS, by quantifying the residual precursors and the impurities still present after synthesis. These quantified impurities are allyl alcohol, hereafter abbreviated AOH, which can be present/form from allyl chloride and SO₃²⁻ precursors for SAS.

TABLE 5
(CE = counter-example)

| | | wt % | Residual | SO₃²⁻ |
|---|---|---|---|---|
| | Origin | of ¹⁴C | AOH | residual |
| | ACL | of ACL | ppm | ppm |
| CE 1 | Fossil | 0 | 250 | 12346 |
| M1 | Non-fossil (tall oil) | 30 | 220 | 4210 |
| M2 | Non-fossil (vegetable oil) | 70 | 158 | 2222 |
| M3 | Non-fossil (tall oil) | 80 | 123 | 2166 |
| M4 | Non-fossil (tall oil) | 100 | 80 | 1498 |
| M5 | Non-fossil (Biomass) | 100 | 85 | 1547 |
| M6 | Non-fossil (vegetable oil) | 100 | 84 | 1632 |
| M7 | Non-fossil (CO₂ capture) | 100 | 82 | 1589 |

The applicant observes that the renewable and non-fossil origin of the allyl chlorides makes it possible to reduce, in the synthesis of SAS, the quantity of SO₃²⁻ residues and to limit the presence/formation of AOH.

### Example 2: Synthesis of Partially Bio-Sourced Copolymer

Two hundred g of deionized water, 700 g of SAS in solution and 142 g of solid maleic anhydride are added to a 1000 mL jacketed reactor, equipped with a condenser and a stirrer.

In this example, the maleic anhydride is not of bio-sourced origin.

The resulting solution is heated to 110, nitrogen bubbling is carried out for 30 minutes to remove all traces of dissolved oxygen.

A persulfate solution is prepared in a separating funnel, by dissolving 25 g of sodium persulfate in 70 g of deionized water. The polymerization initiator solution is added over a period of 300 minutes. After the addition of the persulfate solution, the reaction medium is maintained at 80° C. for 60 minutes; a fluid liquid is obtained.

TABLE 6

| | Polymer | | | | | | | |
|---|---|---|---|---|---|---|---|---|
| | P1 | P2 | P3 | P4 | P5 | P6 | P7 | CE |
| | Monomer | | | | | | | |
| | M1 | M2 | M3 | M4 | M5 | M6 | M7 | CE1 |
| wt % ¹⁴C of | 30 | 70 | 80 | 100 | 100 | 100 | 100 | 0 |
| SAS | | | | | | | | |
| wt % ¹⁴C of | 0 | 0 | 0 | 0 | 0 | 0 | 0 | 0 |
| maleic acid | | | | | | | | |
| wt % ¹⁴C of | 13 | 30 | 34 | 43 | 43 | 43 | 43 | 0 |
| the polymer | | | | | | | | |
| obtained | | | | | | | | |

(CE = counter-example)

### Example 3: Synthesis of Bio-Sourced Co-Polymer

The polymerization protocol described above is reproduced with the difference that the maleic anhydride is of bio-sourced origin.

TABLE 7

| | Polymer | | | | | | | |
|---|---|---|---|---|---|---|---|---|
| | P8 | P9 | P10 | P11 | P12 | P13 | P14 | CE3 |
| | Monomer | | | | | | | |
| | M1 | M2 | M3 | M5 | M5 | M6 | M7 | CE1 |
| wt % ¹⁴C of | 30 | 70 | 80 | 100 | 100 | 100 | 100 | 0 |
| SAS | | | | | | | | |
| wt % ¹⁴C of | 100 | 100 | 100 | 100 | 100 | 100 | 100 | 100 |
| maleic acid | | | | | | | | |
| wt % ¹⁴C of | 70 | 87 | 91 | 100 | 100 | 100 | 100 | 57 |
| the polymer | | | | | | | | |
| obtained | | | | | | | | |

(CE = counter-example)

### Example 4: Antiscale Action of the Copolymers

Static tests of barium sulfate precipitation inhibitions are carried out in the presence of polymers P1 to P14 and of CE 1 and 2 produced in the preceding examples to judge their effectiveness as an antiscaling agent for the inhibition of barium sulfate.

The experimental conditions are as follows:
A solution A containing 0.3518 g/l of barium chloride dihydrate (corresponding to 98.9 ppm of Ba²⁺ ion in the final 100 ml solution) and 40 g/l of NaCl
A solution B containing 0.8 g/l of sodium sulfate (corresponding to 270.5 ppm of SO4²⁻ ion in the final 100 ml solution)
A solution C of acetic acid at a concentration of 60 g/l
A solution D of sodium acetate at a concentration of 136.1 g/l
A solution E containing 0.1 wt % of the above polymers

The test consists of mixing 50 mL of solution A, with 0.1 mL of solution C and 10 mL of solution D, in a 100 mL Nalgene bottle thermostated in a water bath at 35° C. 0.6 mL of solution E are then added, followed by 50 mL of solution B.

The resulting 100 ml of the mixture are placed in an oven at 35° C. for 24 hours for maturation, and are then filtered.

The filtrate is analyzed by atomic absorption to quantify the amount of barium present.

The results are then expressed as a percentage of inhibition according to the following formula, after any correction of the dilution factor necessary for the atomic absorption analysis:

$$Inhibit ( \% ) = \frac{( Ba 2 + ( ppm ) - Ba ++ white ( ppm )}{( 98.9 - Ba ++ white ( ppm ) )} * 100$$

The results are expressed in Table 8.

TABLE 8

| Polymer | % inhibition |
|---|---|
| — | 0 |
| CE 2 | 65 |
| CE 3 | 67 |
| P1 | 86 |
| P2 | 89 |
| P3 | 88 |
| P4 | 96 |
| P5 | 95 |
| P6 | 94 |
| P7 | 94 |
| P8 | 89 |
| P9 | 93 |
| P10 | 94 |
| P11 | 96 |
| P12 | 97 |
| P13 | 95 |
| P14 | 96 |

The Applicant observes that the partially or totally bio-sourced copolymers are more effective as agents for inhibiting scales than the polymers of the prior art (cf. counter-examples). Without wishing to be bound by any theory, the Applicant puts forward the possibility that the different nature of the impurities between a compound of fossil origin and a compound of renewable and non-fossil origin is the cause of these unexpected technical effects.

1. A method for obtaining a bio-sourced (meth)allylsulfonate alkali salt obtained from a (meth)allyl halide and a sulfite ion precursor SO₃²⁻, said (meth)allyl halide being at least partially renewable and non-fossil.

2. The method according to claim 1, wherein the (meth)allyl halide is selected from formula (1) compounds. 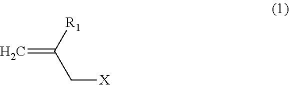
wherein R₁═H or CH₃, and X═Cl, Br, or I.

3. The method according to claim 1, wherein the method comprises a reaction between a (meth)allyl halide that is at least partially renewable and non-fossil, and a sulfite ion precursor SO₃²⁻.

4. The method according to claim 1, wherein the sulfite ion precursor is selected from the group consisting of sodium sulfite Na₂SO₃, potassium sulfite K₂SO₃, sodium bisulfite HNaSO₃, potassium bisulfite HKSO₃, sodium metabisulfite Na₂S₂O₅²⁻, sodium dithionite Na₂S₂O₄, potassium metabisulfite K₂S₂O₅²⁻, potassium dithionite K₂S₂O₄, zinc dithionite ZnS₂O₄, calcium dithionite CaS₂O₄, barium dithionite BaS₂O₄, magnesium dithionite MgS₂O₄, preferentially sodium sulfite or sodium bisulfite.

5. The method according to claim 1, wherein the monomer obtained according to the method is bio-sourced methallylsulfonate sodium salt, bio-sourced allylsulfonate sodium salt, bio-sourced allylsulfonate potassium salt or bio-sourced methallylsulfonate potassium salt.

6. The method according to claim 1, wherein the bio-sourced (meth)allylsulfonate alkali salt has a bio-sourced carbon content of between 5 wt % and 100 wt % relative to the total carbon weight in said bio-sourced (meth)allylsulfonate alkali salt, the bio-sourced carbon content being measured according to the standard ASTM D6866-21 Method B.

7. The method according to claim 1, wherein the (meth)allyl halide has a bio-sourced carbon content of between 5 wt % and 100 wt % relative to the total carbon weight in said (meth)allyl halide, the bio-sourced carbon content being measured according to the standard ASTM D6866-21 Method B.

8. The method according to claim 1, wherein the (meth)allyl halide is partially segregated or totally segregated.

9. The method according to claim 1, wherein the (meth)allyl halide is partially or totally derived from a recycling process.

10. A bio-sourced (meth)allylsulfonate alkali salt obtained from a (meth)allyl halide, said (meth)allyl halide being at least partially renewable and non-fossil.

11. The bio-sourced (meth)allylsulfonate alkali salt according to claim 10, wherein the (meth)allyl halide has a bio-sourced carbon content of between 5 wt % and 100 wt % relative to the total carbon weight in said (meth)allyl halide, the bio-sourced carbon content being measured according to the standard ASTM D6866-21 Method B.

12. A polymer obtained by polymerization of at least one monomer obtained by the method according to claim 1, wherein the polymer is a polymer of:
at least a first monomer obtained by the method according to claim 1, and
at least a second monomer different from the first monomer, said second monomer selected from the group consisting of nonionic monomers, anionic monomers, cationic monomers, zwitterionic monomers, monomers comprising a hydrophobic moiety, and mixtures thereof.

13. (canceled)

14. The polymer according to claim 12, wherein the second monomer is maleic anhydride or its hydrated form, maleic acid.

15. The polymer according to claim 12, wherein the polymer is a polymer comprising:
at least 5 mol %, preferably at least 10 mol %, preferentially between 20 mol % and 90 mol %, more preferentially between 30 mol % and 99 mol % of a first monomer, said monomer being a monomer obtained by the method according to claim 1, and
at least 1 mol %, preferentially between 5 mol % and 90 mol %, more preferentially between 10 mol % and 80 mol % of at least one second monomer comprising an ethylenic unsaturation, said second monomer being different from the first monomer, and comprising a bio-sourced carbon content ranging between 5 wt % and 100 wt %, preferably 10 wt % and 100 wt %, relative to the total carbon content in said second monomer, the bio-sourced carbon content being measured according to the standard ASTM D6866-21 Method B.

16. The polymer according to claim 15, wherein the at least second monomer is selected from the group consisting of acrylamide, acrylic acid, an oligomer of acrylic acid, 2-acrylamido-2-methylpropane sulfonic acid (ATBS) and/or a salt thereof, N-vinylformamide (NVF), N-vinylpyrrolidone (NVP), dimethyldiallylammonium chloride (DADMAC) quaternized dimethylaminoethyl acrylate (ADAME), quaternized dimethylaminoethyl methacrylate (MADAME), a substituted acrylamide having the formula CH₂═CHCO—NR₁R₂, R₁ and R₂ being, independently of each other, a linear or branched carbon chain CₙH₂ₙ₊₁, wherein n ranges between 1 and 10.

17. (canceled)

18. (canceled)

19. (canceled)

20. A method for enhanced oil and/or gas recovery by sweeping a subterranean formation, comprising:
a. preparing an injection fluid from the polymer according to claim 12, with water or brine,
b. injecting the injection fluid into a subterranean formation,
c. sweeping the subterranean formation with the injected fluid,
d. recovering an aqueous mixture of oil and/or gas.

21. A method for hydraulic fracturing of subterranean oil and/or gas reservoirs, comprising:
a. preparing an injection fluid from the polymer, according to claim 12, with water or brine, and with at least one proppant,
b. injecting said fluid into the subterranean reservoir and fracturing at least a portion thereof to recover oil and/or gas.

22. A method of drilling and/or cementing a well in a subterranean formation, comprising:
a. preparing a fluid from the polymer according to claim 12, with water or brine,
b. injecting said drilling and/or cementing fluid into the subterranean formation via the drill head in at least one step of drilling or cementing a well.

23. (canceled)

24. (canceled)

25. (canceled)

26. (canceled)

27. A method for treating a suspension of solid particles in water resulting from mining or oil sands operations, comprising contacting said suspension with at least one polymer according to claim 12.

28. A polymer obtained by polymerization of at least one bio-sourced (meth)allylsulfonate alkali salt monomer according to claim 10.
